(19) United States
(12) Patent Application Publication
(10) Pub. No.: US 2008/0077127 A1
(43) Pub. Date: Mar. 27, 2008

(54) Intraocular pressure control

(76) Inventors: Shawn X. Gao, Irvine, CA (US); Mark A. Hopkins, Mission Viejo, CA (US); David L. Williams, Newport Beach, CA (US)

Correspondence Address: ALCON, IP LEGAL, TB4-8, 6201 SOUTH FREEWAY, FORT WORTH, TX, 76134, US

(21) Appl. No.: 11/830,043

(22) Filed: Jul. 30, 2007

Related U.S. Application Data
(60) Provisional application No. 60/847,438, filed on Sep. 27, 2006.

(51) Int. Cl. A61B 18/04; A61B 5/00

(52) U.S. Cl. 606/34; 600/561

(57) ABSTRACT

An improved method for controlling intraocular pressure during ophthalmic surgery.

This application claims the priority of U.S. Provisional Application Ser. No. 60/847,438 filed on Sep. 27, 2006.

### FIELD OF THE INVENTION

The present invention generally pertains to microsurgical systems and more particularly to controlling intraocular pressure in ophthalmic surgery.

### DESCRIPTION OF THE RELATED ART

During small incision surgery, and particularly during ophthalmic surgery, small probes are inserted into the operative site to cut, remove, or otherwise manipulate tissue. During these surgical procedures, fluid is typically infused into the eye, and the infusion fluid and tissue are aspirated from the surgical site.

Maintaining an optimum intraocular pressure during ophthalmic surgery is currently problematic. When no aspiration is occurring, the pressure in the eye becomes the pressure of the fluid being infused into the eye. This pressure is typically referred to as the “dead head pressure”. However, when aspiration is applied, the intraocular pressure drops dramatically from the dead head pressure due to all the pressure losses in the aspiration circuit associated with aspiration flow. Therefore, ophthalmic surgeons currently tolerate higher than desired dead head pressures to compensate for occasions when aspiration would otherwise lower the intraocular pressure to soft-eye conditions. Clinically, such over-pressurizing of the eye is not ideal.

Accordingly, a need continues to exist for improved apparatus for controlling intraocular pressure during ophthalmic surgery.

### SUMMARY OF THE INVENTION

In one aspect, the present invention is a method of controlling intraocular pressure with a microsurgical system. A desired intraocular pressure is provided, and a known volume of a pressurized gas is stored in a receiver. A pressure transducer for measuring the pressure of the gas in the receiver is also provided. The gas is used to pressurize a surgical fluid stored in an infusion chamber of an ophthalmic surgical cassette. The cassette has a fluid level sensor for measuring the level of the fluid in the infusion chamber. Using a computer, an expected pressure decay in the receiver is calculated using the measured level of the fluid. The computer also calculates a volume flow rate of the fluid using the expected pressure decay, and an expected intraocular pressure using the volume flow rate and the measured pressure of the gas. The amount of the gas used to pressurize the surgical fluid in the infusion chamber is adjusted with the computer based on a comparison of the expected intraocular pressure and the desired intraocular pressure.

### BRIEF DESCRIPTION OF THE DRAWINGS

For a more complete understanding of the present invention, and for further objects and advantages thereof, reference is made to the following description taken in conjunction with the accompanying drawings, in which:

FIG. 1 is a schematic diagram illustrating intraocular pressure control in an ophthalmic microsurgical system;

FIG. 2 is a front, perspective view of a preferred surgical cassette for use in the ophthalmic microsurgical system of FIGS. 1 and 3; and

FIG. 3 is a schematic diagram illustrating intraocular pressure control in an ophthalmic microsurgical system with dual pressurized gas receivers.

### DETAILED DESCRIPTION OF THE PREFERRED EMBODIMENTS

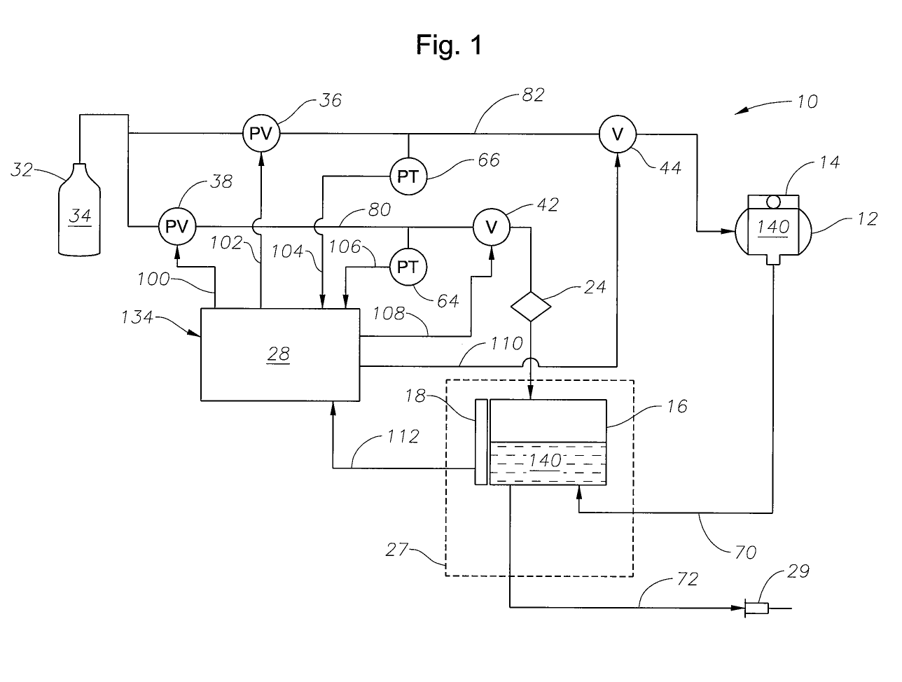

The preferred embodiments of the present invention and their advantages are best understood by referring to FIGS. 1-3 of the drawings, like numerals being used for like and corresponding parts of the various drawings. As shown in FIG. 1, ophthalmic microsurgical system 10 includes a pressure cuff 12, an infusion source 14, an infusion chamber 16, fluid level sensor 18, a filter 24, a surgical device 29, a computer or microprocessor 28, a receiver 32, proportional solenoid valves 36 and 38, “on/off” solenoid valves 42 and 44, and pressure transducers 64 and 66. Receiver 32 contains a pressurized gas 34, preferably air. Infusion chamber 16, fluid level sensor 18; portions of infusion fluid lines 70 and 72, and portions of a gas line 80 are preferably disposed in a surgical cassette 27. Infusion source 14, infusion chamber 16, and surgical device 29 are fluidly coupled via infusion fluid lines 70 and 72. Infusion source 14, infusion chamber 16, filter 24, and receiver 32 are fluidly coupled via gas lines 80 and 82. Fluid level sensor 18, microprocessor 28, proportional solenoid valves 36 and 38, on/off solenoid valves 42 and 44, and pressure transducers 64 and 66 are electrically coupled via interfaces 100, 102, 104, 106, 108, 110, 112.

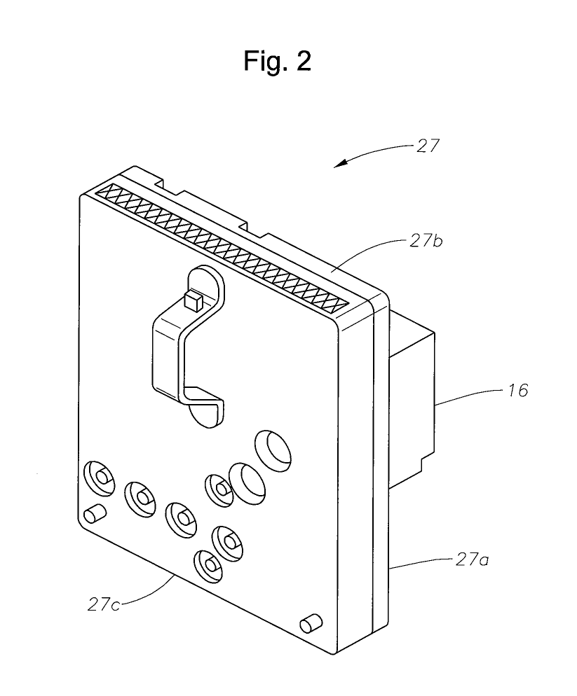

Infusion source 14 is preferably a flexible infusion source. As shown best in FIG. 2, infusion chamber 16 is preferably formed on a rear surface 27a of surgical cassette 27. Surgical cassette 27 preferably also has a top surface 27b and a bottom surface 27c. Fluid level sensor 18 may be any suitable device for measuring the level of fluid in infusion chamber 16. Fluid level sensor 18 is preferably capable of measuring the level of fluid in infusion chamber 16 in a continuous manner. Filter 24 is a hydrophobic micro-bacterial filter. A preferred filter is the Versapor® membrane filter (0.8 micron) available from Pall Corporation of East Hills, N.Y. Microprocessor 28 is capable of implementing feedback control, and preferably PID control. Surgical device 29 may be any suitable device for providing surgical irrigating fluid to the eye but is preferably an infusion cannula, an irrigation handpiece, or and irrigation/aspiration handpiece. The portions of fluid lines 70 and 72 disposed in surgical cassette 27, and the portion of gas line 80 disposed in surgical cassette 27, may be any suitable line, tubing, or manifold for transporting a fluid but are preferably manifolds integrally molded into surgical cassette 27.

In operation, fluid line 70, chamber 16, fluid line 72, and surgical device 29 are all primed with a surgical irrigating fluid 140 by pressurizing infusion source 14. Surgical irrigating fluid 140 may be any surgical irrigating fluid suitable for ophthalmic use, such as, by way of example, BSS PLUS® intraocular irrigating solution available from Alcon Laboratories, Inc.

The pressurizing of infusion source 14 is preferably performed by pressure cuff 12. More specifically, microprocessor 28 sends a control signal to open solenoid valve 44 via interface 110 and to close solenoid valve 42 via interface 108. Microprocessor 28 also sends a control signal to open proportional solenoid valve 36 via interface 102 so that receiver 32 supplies the appropriate amount of pressurized air to actuate pressure cuff 12. Pressure transducer 66 senses the pressure within gas line 82 and provides a corresponding signal to microprocessor 28 via interface 104. Alternatively, the pressuring of infusion source 14 may be performed solely via gravity.

After priming, a user then provides a desired intraocular pressure to microprocessor 28 via an input 134. Input 134 may be any suitable input device but is preferably a touch screen display or physical knob. Microprocessor 28 sends appropriate control signals to open solenoid valve 42 (via interface 108) and to open proportional solenoid valve 38 (via interface 100) to provide an appropriate level of pressurized air to infusion chamber 16. Pressure transducer 64 senses the pressure within gas line 80 and provides a corresponding signal to microprocessor 28 via interface 106. Infusion chamber 16 supplies pressurized fluid 140 to the eye via fluid line 72 and surgical device 29. Fluid level sensor 18 senses the level of surgical irrigating fluid 140 within infusion chamber 16 and provides a corresponding signal to microprocessor 28 via interface 112. As the infusion process commences and proceeds, the consumed volume of surgical irrigating fluid 140 in infusion chamber 16 will be replaced by gas 34; hence the pressure in receiver 32 will decay. Microprocessor 28 calculates the expected pressure decay within receiver 32 using the signal from fluid level sensor 18 and the known volume of infusion chamber 16. Microprocessor 28 then calculates the volume change of fluid 140 within infusion chamber 16, as well as the volume flow rate in the infusion circuit, using the signal from pressure transducer 64. Microprocessor 28 then calculates a predicted intraocular pressure according to the formula P=Q·R where Q is the calculated volume flow rate of surgical irrigating fluid and R is the empirically determined impedance information of microsurgical system 10. Microprocessor then sends an appropriate feedback control signal to proportional solenoid valve 38 to maintain the predicted intraocular pressure at or near the desired intraocular pressure during all portions of the surgery.

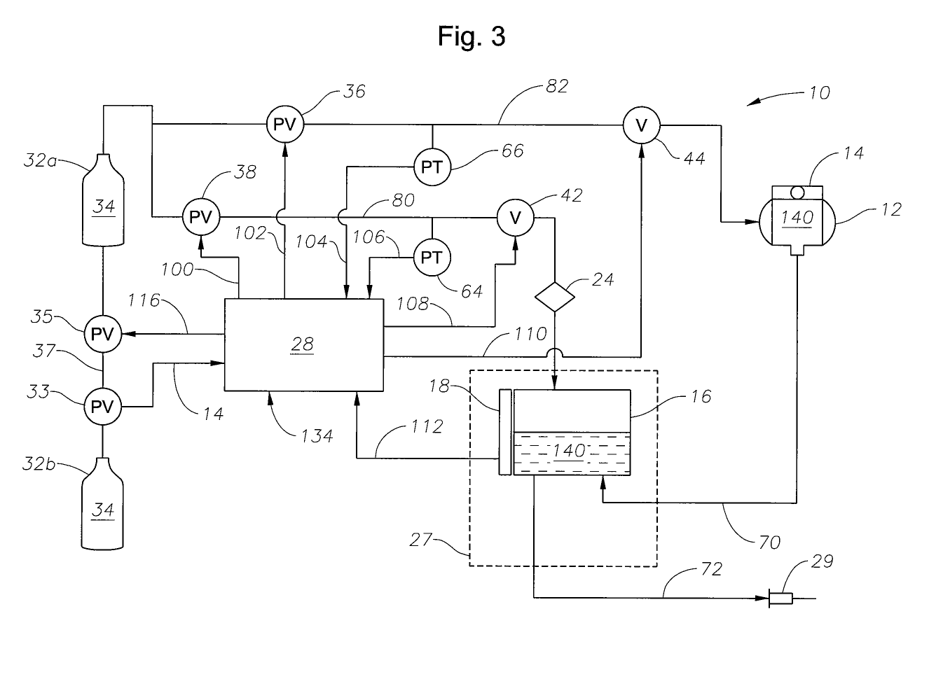

An alternative embodiment to the present invention addresses the problem that, as the infusion process proceeds, the pressure in receiver 32 will decay to the point where it can no longer provide adequate pressure to create flow in the infusion circuit. As shown in FIG. 3, a second receiver 32b containing pressurized gas 34 (preferably air) is added to system 10. The second receiver 32b will be fluidly connected to a pressure transducer 33, a proportional solenoid valve 35, and primary receiver 32a via gas line 37. During operation, as the calculated volume flow rate of surgical irrigating fluid decreases to a predetermined level, microprocessor 28 signals valve 35 to open via interface 116. Pressure transducer 33 measures the pressure in gas line 37 and provides a corresponding signal to microprocessor 28 via interface 114. Microprocessor 28 calculates the expected rise in pressure of primary receiver 32a from the known volume of secondary receiver 32b and the signal from pressure transducer 33. This expected rise in pressure will allow the infusion control algorithm in microprocessor 28 to anticipate a pressure change due to recharging, and adjust properly to maintain a stable infusion circuit pressure.

From the above, it may be appreciated that the present invention provides an improved method of controlling intraocular pressure with a microsurgical system. The present invention is illustrated herein by example, and various modifications may be made by a person of ordinary skill in the art. For example, while the present invention is described above relative to controlling intraocular pressure in an ophthalmic microsurgical system, it is also applicable to controlling pressure within the operative tissue during other types of microsurgery.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims

What is claimed is:

1. A method of controlling intraocular pressure with a microsurgical system, comprising the steps of:
providing a desired intraocular pressure;
storing a known volume of a pressurized gas in a receiver;
providing a pressure transducer for measuring the pressure of said gas in said receiver;
using said gas to pressurize a surgical fluid stored in an infusion chamber of an ophthalmic surgical cassette, said cassette having a fluid level sensor for measuring the level of said fluid in said infusion chamber;
calculating an expected pressure decay in said receiver with a computer using said measured level of said fluid;
calculating a volume flow rate of said fluid with said computer using said expected pressure decay;
calculating an expected intraocular pressure with said computer using said volume flow rate and said measured pressure of said gas; and
adjusting said amount of said gas in said using step with said computer based on a comparison of said expected intraocular pressure and said desired intraocular pressure.

2. The method of claim 1 wherein said gas is air.

3. The method of claim 1 further comprising the steps of:
providing a second receiver containing a known volume of said gas; and
using said second receiver to provide additional gas to said receiver.
